(19) United States
(12) Patent Application Publication
(10) Pub. No.: US 2010/0130946 A1
(43) Pub. Date: May 27, 2010

(54) Heel cap for skin treatment

(76) Inventor: Geraldine Price Medley, Gaffney, SC (US)

Correspondence Address: J. BENNETT MULLINAX, LLC, P. O. BOX 26029, GREENVILLE, SC, 29616-1029, US

(21) Appl. No.: 12/313,862

(22) Filed: Nov. 25, 2008

(51) Int. Cl. A61M 35/00

(52) U.S. Cl. 604/293

(57) ABSTRACT

A heel cap is provided. The heel cap includes a body having an outer surface and an inner surface. At least a portion of the outer surface and the inner surface are made of the same type of plastic. The body has a back wall, a left side wall, a right side wall, and a bottom wall. The body has an open top and an open front and is configured for being attached to the heel of a user.

### FIELD OF THE INVENTION

The present invention relates generally to a heel cap for use in therapeutic treatment of the skin of the user. More particularly, the present application involves a heel cap for use in containing therapeutic lotions and creams onto the heel of the user while preventing such therapeutic applications from being inadvertently spilled or otherwise transferred to the surrounding environment.

### BACKGROUND

Topical applications are often employed in the treatment of skin ailments that effect the skin of individuals. For example, the heels on the feet of individuals often become dry or cracked thus requiring the application of an ointment thereon for correction. Cracked heels can be caused by dry skin in combination with other factors. For example, prolonged standing, existing medical conditions, shoe types, and being overweight may also cause cracked heels. In certain cases, cracked heels amount to nothing more than a cosmetic problem. However, in other instances cracked heels may lead to long, deep fissures that can cause significant pain and bleeding which may lead to infections.

Treatment of dry or cracked heels often involves the application of a moisturizing agent thereon in order to reduce dryness and promote healing. The topical application may be an oil based moisturizing cream, a lotion that includes glycolic or lactic acid, or can be any of a variety of ointments. Once applied, the topical application must remain on the heel of the user for a period of time in order to allow same to be absorbed therein to deliver its intended benefits. The heel typically has a layer of thickened, callused skin thereon which acts to prevent topical applications from being absorbed. As such, the user must avoid contact between his or her heel and the floor, bed, couch, clothing or other items to prevent the topical application from being removed.

Various methods have been employed in order to maintain a topical application onto the heel of a user. For example, plastic wrap may be employed to cover the topical application when applied. Such a technique may be problematic in that the plastic wrap can be relatively easily moved out of position should the user move his or her heel against an object. Also, placement of a sock over the topically applied heel has been employed. Aside from staining the sock, the topical application may flow out of the sock and away from the heel of the user since a sock is not a fluid impervious object.

Wraps are known that include cotton gauze applicators for topical applications that are configured in such a manner to allow a sock to be worn over the wrap and applicator. Aside from allowing the topical application to flow therethrough, wraps of this type are configured in a manner that requires the user possess a great degree of dexterity in attaching the wrap to his or her heel. Other types of footwear include a slipper that has a multi-layered insert that includes a topical application. The entire foot of the user is contained within the slipper so that the foot is kept warm to enhance treatment. Further, the complete containment of the foot within the footwear prevents the topical application from flowing to the outside of the footwear. Although suitable for their intended purpose, slippers of this type are not reusable with other topical applications and are uncomfortable to wear when sleeping. As such, there remains room for variation and improvement within the art.

### BRIEF DESCRIPTION OF THE DRAWINGS

A full and enabling disclosure of the present invention, including the best mode thereof, directed to one of ordinary skill in the art, is set forth more particularly in the remainder of the specification, which makes reference to the appended Figs. in which:

FIG. 1 is a perspective view of a heel cap in accordance with one exemplary embodiment.

FIG. 2 is a side view of the heel cap of FIG. 1.

FIG. 3 is a top view of the heel cap of FIG. 1.

FIG. 4 is a cross-sectional view taken along line 4-4 of FIG. 3.

FIG. 5 is a front view of the heel cap of FIG. 1.

FIG. 6 is a bottom view of the heel cap of FIG. 1.

FIG. 7 is a side view of the heel cap of FIG. 1 shown attached to the heel of a user.

Repeat use of reference characters in the present specification and drawings is intended to represent the same or analogous features or elements of the invention.

### DETAILED DESCRIPTION OF REPRESENTATIVE EMBODIMENTS

Reference will now be made in detail to embodiments of the invention, one or more examples of which are illustrated in the drawings. Each example is provided by way of explanation of the invention, and not meant as a limitation of the invention. For example, features illustrated or described as part of one embodiment can be used with another embodiment to yield still a third embodiment. It is intended that the present invention include these and other modifications and variations.

It is to be understood that the ranges mentioned herein include all ranges located within the prescribed range. As such, all ranges mentioned herein include all sub-ranges included in the mentioned ranges. For instance, a range from 100-200 also includes ranges from 110-150, 170-190, and 153-162. Further, all limits mentioned herein include all other limits included in the mentioned limits. For instance, a limit of up to 7 also includes a limit of up to 5, up to 3, and up to 4.5.

The present invention provides for a heel cap 10 for use in applying a topical application 74 onto the heel 64 of a user 30 in order to treat skin conditions associated with the heel 64. The heel cap 10 prevents the topical application 74 from being wiped away from the heel 64 and onto covers when the user sleeps or onto the floor when the user walks. The heel cap 10 may have a body 12 that is made from plastic, such as polyvinyl chloride, and can be rigid enough so that left and right side walls 20 and 22 of the body 12 are formed and extend vertically from a bottom wall 24 of the body 12 when the body 12 is not attached to the user 30. In this manner, the user 30 may more easily place his or her heel 64 into the body 12 when attaching the heel cap 10.

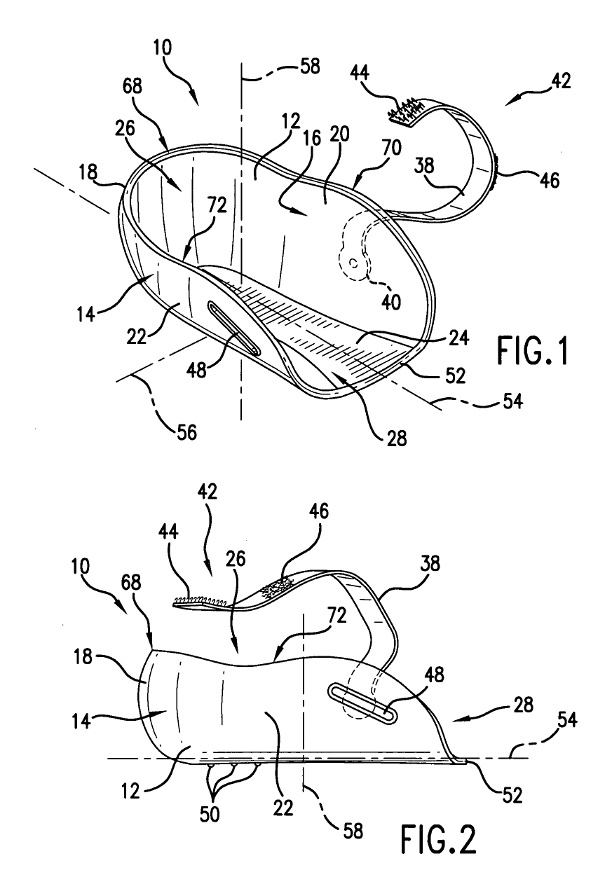

An exemplary embodiment of the heel cap 10 is illustrated in FIGS. 1-6 in which the heel cap 10 is not attached to a heel 64 of a user 30. The heel cap 10 includes a body 12 that may be a single, molded integral piece in accordance with certain exemplary embodiments. In this regard, the body 12 can be a single piece of plastic, such as polyvinyl chloride, and may be made through a molding process. In accordance with one exemplary embodiment, the body 12 is made of APEX® 3210-60 flexible polyvinyl chloride provided by the Teknor Apex Company having offices at 505 Central Avenue, Pawtucket, R.I., 02861, USA. This polyvinyl chloride material may have a tensile strength at 100% strain of 480 psi, a tensile strength at break of 1200 psi, and a tensile elongation at break of 390% all evaluated under an ASTM D638 test method. This polyvinyl chloride material may have a durometer hardness of Shore A at 15 seconds of 60 as evaluated under the ASTM D2240 test method. This material may also have a specific gravity of 1.35 as measured under the ASTM D792 test method, and a brittleness temperature of −36.4° F. as measured under the ASTM D746 test method.

In accordance with other exemplary embodiments, the material that makes up the body 12 may have a tensile strength at 100% strain from 440-520 psi, the tensile strength at break from 1100-1300 psi, and the tensile elongation at break from 370% to 420% all evaluated under the ASTM D638 test method. Further, the material included within body 12 may have a durometer hardness of Shore A at 15 seconds from 50-70. Also, the material may have a specific gravity of 1.20-1.5 as measured under the ASTM D792 test method, and a brittleness temperature of −40° F. to −30° F. as measured under the ASTM D746 test method. Also, although described as being a single piece of molded polyvinyl chloride, it is to be understood that the body 12 can be made of other types of plastic such as polyethylene terephthalate, high-density polyethylene, low-density polyethylene, polypropylene, and polystyrene. The plastic making up the body 12 can be medical grade flexible plastic in accordance with certain exemplary embodiments. Further, the body 12 need not be made out of a single type of plastic but may be made of multiple types of plastic in accordance with other exemplary embodiments. Still further, the body 12 may include other materials, such as fabric, metal, or hook and loop type fasteners, which are attached to the plastic.

The body 12 has an outer surface 14 and an inner surface 16. The body 12 can be a single integral piece so that the body 12 is a single layer such that the entire outer surface 14 and inner surface 16 are made from the same material. Alternatively, the body 12 can be constructed so that only a portion of the outer surface 14 and/or inner surface 16 are made of plastic. The body 12 is arranged so as to form a back wall 18, a left side wall 20, a right side wall 22, and a bottom wall 24. The back wall 18, left side wall 20 and right side wall 22 extend upwards from the bottom wall 24 in the vertical direction. The vertical direction of the body extends in the direction of the vertical axis 58. The walls 18, 20 and 22 have enough rigidity so that they maintain their vertical extension from the bottom wall 24 when the body 12 is not attached to the heel 64 of the user 30. As such, the body 12 is a substantially rigid structure capable of maintaining its shape when placed onto the floor or when turned onto its side. However, in certain embodiments the walls 18, 20 and 22 are sufficiently flexible so that they can flex with respect to the bottom wall 24 when the user 30 places his or her heel 64 into the body 12. In this regard, the walls 18, 20 and 22 are capable of flexing so that the volume of the body 12 can expand in order to accommodate different sized heels 64 of different users 30. In this way, the heel cap 10 can be provided in a particular size that accommodates variously sized users 30.

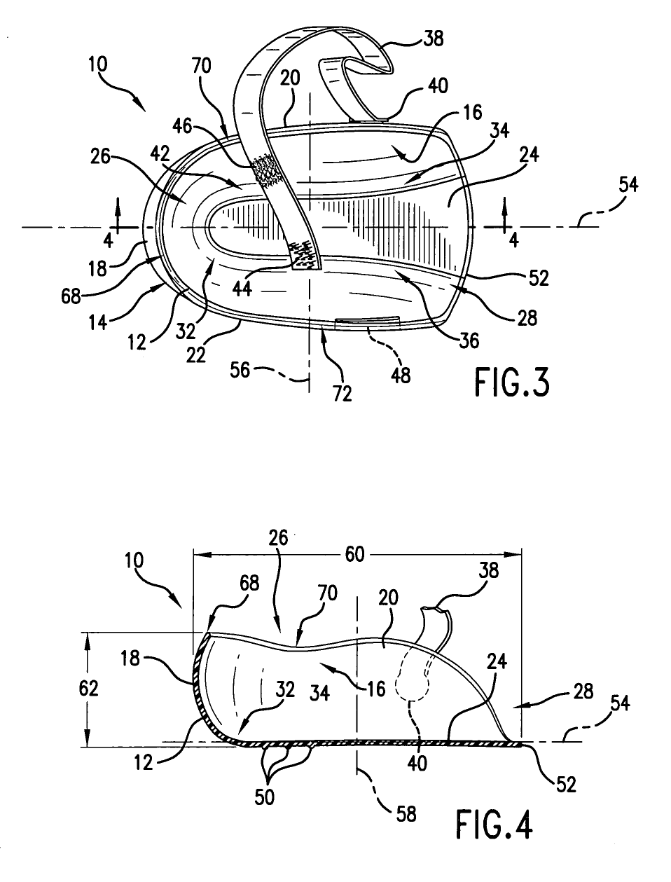

The inner surface 16 of the bottom wall 24 may be planar in certain embodiments. As shown in the top view of FIG. 3, the inner surface 16 of the bottom wall 24 is irregular in shape. However, the inner surface 16 of the bottom wall 24 may be rectangular in shape in accordance with other exemplary embodiments. The bottom wall 24 has a leading edge 52 that is located at the forward most location of the body 12 along the longitudinal axis 54. The back wall 18 is located on the side of the bottom wall 24 that is opposite the leading edge 52 in the longitudinal direction. The back wall 18 may be defined as having a width in the lateral direction, that is in the direction along the lateral axis 56, which is the same as the point of greatest width in the lateral direction of the bottom wall 24. In the exemplary embodiment shown, the greatest width of the bottom wall 24 in the lateral direction is at the leading edge 52. As such, the back wall 18 has a width in the lateral direction that is the same as the width of the leading edge 52 in the lateral direction. In accordance with other exemplary embodiments, the bottom wall 24 may be rectangular in shape so that the back wall 18 has a width in the lateral direction that is essentially the same as the width of the bottom wall 24 in the lateral direction.

As shown with reference to FIGS. 2, 4 and 7 the bottom wall 24 includes a slight arch such that the inner surface 16 and outer surface of the bottom wall 24 arch upwards near the center of the bottom wall 24 in the longitudinal direction. Such arch may assist in the retention of topical application 74 within the heel cap 10 and prevent same from leaking across the leading edge 52 and out of the heel cap 10. The arch of the bottom wall 24 can be present at times when the heel cap 10 is not worn by the user and present at times when the heel cap 10 is worn by the user. The arch may be arranged in certain embodiments in which the portion of the inner surface 16 on one side of the arch in the longitudinal direction has the same height in the lateral direction as the portion of the inner surface 16 near the leading edge 52 that is located on the other side of the arch in the longitudinal direction. Although described as including a slight arch, the bottom wall 24 need not include such arch in accordance with other exemplary embodiments of the heel cap 10.

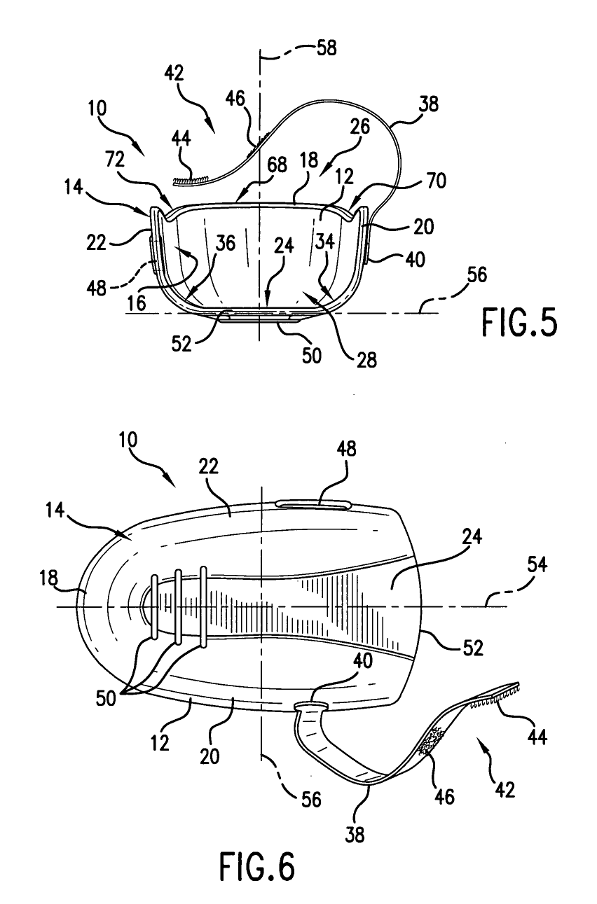

A portion 32 of the back wall 18 is attached to the bottom wall 24 and is curved or concave in shape. Other portions of the back wall 18 besides portion 32 may be planar or curved in shape. As shown with reference to FIG. 4, the entire inner surface 16 of the back wall 18 is concave in shape. The concave shape of the back wall 18 may allow for a more comfortable and secure fit and receipt of the back of the heel 64 due to their complimentary curvature. With reference now to FIG. 5, the inner surface 16 of the left side wall 20 has a portion 34 that extends from the bottom wall 24. Portion 34 is concaved or curved in shape. The remaining portions of the left side wall 20 are both concave and planar in shape. In accordance with other exemplary embodiments, the entire inner surface 16 of the left side wall 20 may be planar or concave in shape. The right side wall 22 also has a portion 36 on its inner surface 16 that is curved or concave in shape and is attached to the bottom wall 24. The remaining portions of the inner surface 16 of the right side wall 22 may also be planar or concave in other exemplary embodiments. As disclosed, the inner surface 16 of the body 12 is curved or concave in shape around the entire perimeter of the inner surface 16 of the bottom wall 24 except for around the leading edge 52 which forms an exterior boundary of the body 12. As shown with reference to FIG. 5, the inner surfaces 16 of the left side wall 20 and the right side wall 22 proximate to the upper edges 70 and 72 are not co-planar with one another along the forward half of the left and right side walls 20 and 22 in the longitudinal direction.

The body 12 may be constructed so that its length is sufficient to cover a sizable amount of the foot of the user 30 in the longitudinal direction while covering a smaller amount of the foot in the vertical direction. As shown with reference to FIG. 4, the body 12 has an overall length that is represented by distance 60. Distance 60 extends from the leading edge 52 of the bottom wall 24 to the most remote portion of the outer surface 14 of the back wall 18 in the longitudinal direction. As previously discussed, the longitudinal direction extends in the same direction as the longitudinal axis 54. Distance 60 may be from 3.5 inches to 5.0 inches in accordance with certain exemplary embodiments. In accordance with one exemplary embodiment, distance 60 is 5.0 inches. However, it is to be understood that distance 60 may be variously sized in accordance with other exemplary embodiments. On most users 30, the length of distance 60 is selected so that the body 12 extends from the heel 64 of the user 30 to a location approximately midpoint of the arch of the foot of the user 30 in the direction from the heel 64 to the toes 66 of the foot.

The body 12 is arranged so as to have a maximum height that extends from the outer surface 14 of the bottom wall 24 to the upper edge 68 of the back wall 18 in the vertical direction which extends in the same direction as the vertical axis 58. The maximum height of the body 12 may extend from the outer surface 14 of the bottom wall 24 to the upper edges 70 and 72 of the left and right side walls 20 and 22 in accordance with other exemplary embodiments if these components of the body 12 are configured differently. The distance 62 may be from 1.50 inches to 2.00 inches in accordance with certain exemplary embodiments. In accordance with one embodiment, distance 62 is 2.00 inches. However, it is to be understood that the distance 62 may be yet a different value in other embodiments. The value of distance 62 may be selected so that the height of the body 12 does not reach the ankle of the user 30 when the body 12 is attached to the heel 64 of the user 30. The upper edges 70 and 72 of the left and right side walls 20 and 22 have an irregular curvature such that they extend towards the bottom wall 24 and then away from the bottom wall 24 in the longitudinal direction extending from the back wall 18 to the leading edge 52. The upper edges 70 and 72 then extend downwards towards the bottom wall 24 and engage the leading edge 52. As shown with reference to FIG. 3, the upper edges 70 and 72 both engage the leading edge 52 of the bottom wall 24 such that the leading edge 52 remains the outer most portion of the body 12 in the longitudinal direction.

The upper edges 68, 70 and 72 define an open top 26 of the body 12. The upper edges 70 and 72 along with the leading edge 52 define an open front 28 of the body 12. The open top 26 and open front 28 are defined when the body 12 is not attached to the heel 64 of the user 30. During attachment, the foot of the user 30 is located within the open top 26 and open front 28.

A strap 38 may be attached to one of the side walls 20 or 22 of the body 12. As shown in FIGS. 1-6, strap 38 is attached to the outer surface 14 of the left side wall 20. The strap 38 may be attached in a variety of manners. For example, the end of strap 38 may be attached to the left side wall 20 through welding, adhesion, molding, or by way of mechanical fasteners. In accordance with one exemplary embodiment, the end of strap 38 is attached through the use of a braid 40 to the left side wall 20. The braid 40 may be arranged so that the strap 38 can pivot with respect to the left side wall 20 or may be made so that it is incapable of pivoting therewith. The braid 40 can include a rivet or a pin that is attached to the left side wall 20 in a pivoting engagement therewith. As such, the strap 38 may be rigidly attached to the left side wall 20 in accordance with certain exemplary embodiments. The strap 38 may be made of a material that is different than the material making up with body 12. For example, the strap 38 may be made of fabric or can be made of a different type of plastic than the plastic making up body 12. However, in certain instances the strap 38 may be made of the same material as the body 12. The strap 38 is constructed so that it enjoys some degree of flexibility.

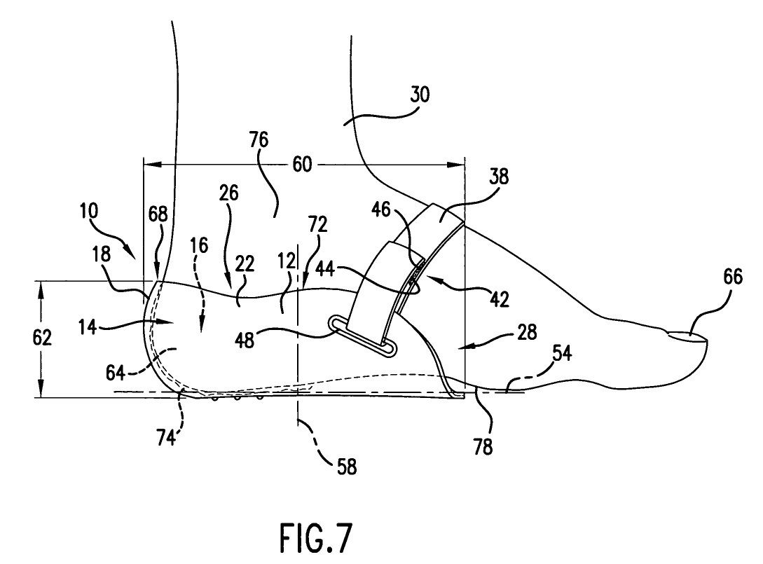

FIG. 7 illustrates the heel cap 10 as attached to the foot of the user 30 in accordance with one exemplary embodiment. In use, the user 30 may apply a topical application 74 to his or her heel 64. Additionally or alternatively, the user 30 may apply the topical application 74 to the inner surface 16 of the body 12. Next, the user 30 can place the heel cap 10 on the floor and may position his or her heel 64 therein. Should the user 30 possess sufficient dexterity, the user 30 may place the heel cap 10 onto his or her heel 64 by manually grasping the heel cap 10 and placing the heel cap 10 into the desired position. The body 12 includes a back wall 18, left side wall 20 and a right side wall 22 that maintain their shape when the heel cap 10 is positioned on the floor so that the user 30 may more easily slide his or her heel 64 into the body 12 without the need to pull any part of the heel cap 10 around his or her foot.

Any combination of the back wall 18, left side wall 20 and right side wall 22 of the body 12 may be sufficiently flexible so that if the foot of the user 30 is wide or larger than the initial volume of the body, the walls 18, 20 and 22 may flex outwards some degree in order to accommodate the user's 30 slightly larger foot. However, it is to be understood that the walls 18, 20 and 22 need not be flexible in accordance with other exemplary embodiments. The foot of the user 30 is positioned within the body 12 so that the heel 64 is contained therein and engages the topical application 74. The body 12 is not provided with an applicator pad in the disclosed embodiment. In this regard, the topical application 74 contacts directly the inner surface 16 of the body 12. However, it is to be understood that an applicator pad may be present in accordance with other exemplary embodiments.

The body 12 has a height in the vertical direction such that the ankle 76 of the user 30 is located above the body 12. In this regard, the ankle 76 does not engage the upper edges 68, 70 or 72. Placement of the body 12 below the ankle 76 affords the user 30 with a greater range of motion and less irritation since the ankle 76 is not constrained by the heel cap 10. The foot of the user 30 extends outwards from the open front 28 of the body 12. The body 12 has a length in the longitudinal direction such that the leading edge 52 is may be located at the midpoint of the arch 78 of the foot of the user 30. However, depending upon the selected length of the body 12 and the particular size of the user's foot, the leading edge 52 may be located at a position other than the midpoint of the arch 78 in other embodiments. Placement of the leading edge 52 at a distance from the heel 64 functions to prevent topical application 74 on the inner surface 16 and heel 64 from escaping due to the extended length of the body 12 in the longitudinal direction. Further, the function of the foot of the user 30 is not significantly impacted since the arch 78 of the user 30 typically does not flex or bend a significant degree during use of the foot. As such, the portion of the foot of the user 30 forward of the leading edge 52 is disposed outside of the body 12.

The strap 38 may be employed in order to securely hold the body 12 to the heel 64 of the user 30. The strap 38 may be positioned over the top of the foot of the user and slid through a side wall aperture 48 formed in the right side wall 22. The side wall aperture 48 may be a slot that is completely disposed through the right side wall 22. The side wall aperture 48 may be located on the left side wall 20 and the strap 38 attached to the right side wall 22 when the heel cap 10 is configured for use on the right foot of the user. However, it is to be understood that the side wall aperture 48 and the attachment point of the strap 38 may be provided either on the right or left side wall 22 or 20 regardless of whether the heel cap 10 is used for the left or right foot of the user in accordance with various exemplary embodiments.

When provided as a slot, the side wall aperture 48 may be arranged at an angle of 25° to the planar inner surface 16 of the bottom wall 24. However, in other exemplary embodiments, the side wall aperture 48 may be at an angle from 15° to 35° to the planar inner surface 16 of the bottom wall 24. The strap 38 is disposed through the side wall aperture 48 and brought back up over and onto itself on the top of the foot of the user 30. The strap 38 includes a hook and loop type fastener 42 thereon. The hook and loop type fastener 42 includes hooks 44 and loops 46 that can engage one another in order to effect attachment of the hook and loop type fastener 42. The user 30 may apply a significant amount of force to the strap 38 so that the left and right side walls 20 and 22 flex inwards. In this regard, the side walls 20 and 22 may be pulled against the sides of the foot of the user 30 to provide a secure attachment. Inward flexing of the side walls 20 and 22 may allow for the body 12 to be more securely attached in instances when the foot of the user 30 is narrower than the initial distance between the left side wall 20 and the right side wall 22. The front of the foot of the user 30 extends outward from the strap 38 and the body 12 so that this portion of the foot is uncovered by the heel cap 10. The hook and loop type fastener 42 may be disengaged and the strap 38 can be pulled through the side wall aperture 48 when the user desires the removal of the heel cap 10.

The heel cap 10 may be worn at night when the user 30 is sleeping so that the topical application 74 remains on the heel 64 of the user 30 and does not stain or otherwise damage covers or bed sheets. The topical application 74 may thus be retained onto the heel 64 for a sufficient time so as to cause absorption therein to obtain beneficial results. Although intended for nighttime sleeping use, it is contemplated that the user 30 will wear the heel cap 10 when walking, such as when using the bathroom at night or at other times before or after sleep. In these circumstances, the heel cap 10 is provided with a plurality of traction elements 50 located on the outer surface 14 of the body 12. The traction elements 50 may be made of the same material as the body 12 and can be integrally formed with the body 12 in instances in which the body 12 is formed through molding. The traction elements 50 can be parallel in formation to one another and may be located on the outer surface 14 of the bottom wall 24. Additionally or alternatively, the traction elements 50 may also be located on the outer surface 14 of the left side wall 20 or right side wall 22. The traction elements 50 may also be located on the outer surface 14 of the back wall 18 in other exemplary embodiments. The traction elements 50 function to provide some degree of traction to the heel cap 10 should the user 30 attempt to walk while wearing the heel cap 10.

When composed of plastic and lacking an applicator pad, the body 12 of the heel cap 10 may be easily washed so that dried topical application 74 and can be removed in order to allow the heel cap 10 to be reused. The construction of the strap 38 may also allow this component to be washed and hence reused as desired. However, it is to be understood that the heel cap 10 may be provided in other embodiments in such a manner so as to be a single use, disposable item. In accordance with certain exemplary embodiments, the inner surface 16 of the bottom wall 24 may have a greater surface area than the inner surface 16 of the back wall 18. In accordance with other exemplary embodiments, a lesser portion of the inner surface 16 of the body 12 contacts the heel of the user 30 than contacts other portions of the foot of the user 30 besides the heel of the user 30.

While the present invention has been described in connection with certain preferred embodiments, it is to be understood that the subject matter encompassed by way of the present invention is not to be limited to those specific embodiments. On the contrary, it is intended for the subject matter of the invention to include all alternatives, modifications and equivalents as can be included within the spirit and scope of the following claims.

What is claimed:

1. A heel cap, comprising:
a body having an outer surface and an inner surface, wherein at least a portion of the outer surface and the inner surface are made of the same type of plastic, wherein the body has a back wall, a left side wall, a right side wall, and a bottom wall, and wherein the body has an open top and an open front, wherein the body is configured for being attached to the heel of a user.

2. The heel cap as set forth in claim 1, wherein the entire inner surface of the body and the entire outer surface of the body are made from the same type of plastic.

3. The heel cap as set forth in claim 1, wherein the type of plastic is polyvinyl chloride.

4. The heel cap as set forth in claim 1, wherein the body is made of a single molded piece of the same type of plastic, wherein the back wall, the left side wall, and the right side wall are present when the heel of the user is unattached to the body, wherein the inner surface of the bottom wall is planar, and wherein the portions of the inner surfaces of the back wall, the left side wall, and the right side wall that are contiguous with the inner surface of the bottom wall are concave.

5. The heel cap as set forth in claim 1, further comprising a strap attached to one of the side walls of the body, wherein the strap has a hook and loop type fastener disposed thereon, wherein the other one of the side walls of the body defines a side wall aperture for use in receiving the strap, wherein the strap is disposed through the side wall aperture and the hook and loop type fastener is secured in order to attach the body to the heel of the user.

6. The heel cap as set forth in claim 5, wherein the left side wall and the right side wall are capable of flexing when the strap is used to secure the body to the heel of the user.

7. The heel cap as set forth in claim 1, further comprising a plurality of traction elements located on the outer surface of the body, wherein the traction elements are configured to provide traction when walking when the body is attached to the heel of the user.

8. The heel cap as set forth in claim 1, wherein the body has a longitudinal axis, and wherein the bottom wall has a leading edge that is opposite the back wall, and wherein the distance from the leading edge of the bottom wall to the most remote exterior portion of the outer surface of the back wall in the direction along the longitudinal axis is from 3.5 inches to 5.0 inches.

9. The heel cap as set forth in claim 8, wherein the leading edge of the bottom wall is completely forward the back wall, the left side wall, and the right side wall along the longitudinal axis, and wherein the inner surface of the bottom wall is planar.

10. A heel cap, comprising:
a body having an outer surface and an inner surface, wherein the body is a single molded integral piece having a back wall, a left side wall, a right side wall, and a bottom wall that are all made from a single layer of plastic that extends from the outer surface to the inner surface of the body, wherein the back wall, the left side wall, and the right side wall are sufficiently rigid so as to extend from the bottom wall in the vertical direction of the body when the body is unattached to the heel of a user and when the body is attached to the heel of the user.

11. The heel cap as set forth in claim 10, wherein the type of plastic that makes up the back wall, the left side wall, the right side wall, and the bottom wall of the body is polyvinyl chloride.

12. The heel cap as set forth in claim 10, wherein the body has an open top and an open front, wherein the foot of the user extends through the open front of the body when the heel cap is attached to the heel of the user.

13. The heel cap as set forth in claim 10, wherein the entire inner surface of the body is made of the plastic that extends from the outer surface to the inner surface of the body, wherein the heel of the user is configured to contact the plastic inner surface of the body when the body is attached to the heel of the user.

14. The heel cap as set forth in claim 10, further comprising a strap attached to one of the side walls of the body, wherein the strap has a hook and loop type fastener disposed thereon, wherein the other one of the side walls of the body defines a side wall aperture for use in receiving the strap, wherein the strap is disposed through the side wall aperture and the hook and loop type fastener is secured in order to effect attachment of the body to the heel of the user, wherein the left side wall and the right side wall are capable of flexing when the strap is secured when effecting attachment of the body to the heel of the user.

15. The heel cap as set forth in claim 10, further comprising a plurality of traction elements located on the outer surface of the body, wherein the traction elements are configured to provide traction when walking when the body is attached to the heel of the user.

16. The heel cap as set forth in claim 10, wherein the body has a longitudinal axis, and wherein the bottom wall has a leading edge that is opposite the back wall, and wherein the distance from the leading edge of the bottom wall to the most remote exterior portion of the outer surface of the back wall in the direction along the longitudinal axis is from 3.5 inches to 5.0 inches.

17. A heel cap, comprising:
a body configured for attachment to the heel of a user, wherein the length of the body is from 3.50 inches to 5.00 inches, wherein the length of the body extends in a direction from the heel of the user to the toes of the user when the body is attached to the heel of the user, wherein the height of the body is from 1.25 inches to 2.00 inches, wherein the height of the body extends in a direction from the bottom of the heel of the user to the ankle of the user when the body is attached to the heel of the user, wherein the body has an open front and an open top, wherein the body has a back wall that has a concave inner surface.

18. The heel cap as set forth in claim 17, wherein the body is a single molded integral piece made of polyvinyl chloride.

19. The heel cap as set forth in claim 18, wherein the body has a left side wall, a right side wall, and a bottom wall, wherein the body is sufficiently rigid such that the back wall, the left side wall, and the right side wall are present when the heel of the user is unattached to the body, wherein the inner surface of the bottom wall is planar, and wherein the portions of the inner surfaces of the back wall, the left side wall, and the right side wall that are contiguous with the inner surface of the bottom wall are concave.

20. The heel cap as set forth in claim 19, wherein the body has a longitudinal axis, and wherein the bottom wall has a leading edge that is opposite the back wall, wherein the leading edge of the bottom wall is completely forward the back wall, the left side wall, and the right side wall along the longitudinal axis; and
further comprising a strap attached to one of the side walls of the body, wherein the strap has a hook and loop type fastener disposed thereon, wherein the other one of the side walls of the body defines a side wall aperture for use in receiving the strap, wherein the strap is disposed through the side wall aperture and the hook and loop type fastener is secured in order to effect attachment of the body to the heel of the user, wherein the left side wall and the right side wall are capable of flexing when the strap is secured when effecting attachment of the body to the heel of the user.
